(19) United States
(12) Patent Application Publication
(10) Pub. No.: US 2024/0108406 A1
(43) Pub. Date: Apr. 4, 2024

(54) RENAL DENERVATION CATHETER

(71) Applicant: NU EYNE CO.,LTD, Seoul (KR)

(72) Inventors: Do-hyoung KIM, Seoul (KR); Won-jang Lee, Seoul (KR)

(21) Appl. No.: 18/553,996

(22) PCT Filed: Apr. 5, 2021
(86) PCT No.: PCT/KR2021/004183

§ 371 Date: Oct. 5, 2023

(51) Int. Cl. A61B 18/14; A61B 18/12

(52) U.S. Cl. CPC A61B 18/1492; A61B 18/1206; A61B 2018/00511

(57) ABSTRACT

The present invention relates to a renal denervation catheter which detects contact of a heating electrode with a renal vessel surface and detects reaction sensitivity of a target nerve by a detection electrode so as to excise only the target nerve. To this end, the renal denervation catheter is disclosed, wherein the renal denervation catheter comprises: a catheter housing; an electrode part provided in a housing, emitting a contact detection RF signal for contact with the inner wall of the renal vessel according to a contact detection mode, and emitting a denervation RF signal for renal denervation according to a denervation mode; a control part for allowing to enter the denervation mode when a contact detection feedback signal value generated by the contact detection mode satisfies a defined condition.

### TECHNICAL FIELD

The present invention relates to a renal denervation catheter and, more specifically, to a renal denervation catheter which detects contact of a heating electrode with a renal vessel surface and detects reaction sensitivity of a target nerve by a detection electrode so as to efficiently excise only the target nerve.

### BACKGROUND ART

As a prior art document, Korean patent KR 10-2033760 discloses a catheter for nerve excision. The prior art patent document discloses a device, in which a housing can be flexibly bent so that a heating electrode disposed in the housing of the catheter comes into contact with an inner wall of a renal vessel as closely as possible.

In order to properly excise a nerve positioned on the outside of the renal vessel, the heating electrode disposed on the catheter inserted inside the renal vessel must be as close as possible to the inner wall of the renal vessel. Although the prior art patent document discloses a technology that can be positioned as close as possible to the blood vessel inner wall using a flexibly bendable device, it does not disclose a technique for determining whether a heating electrode is in contact with an inner wall of a renal vessel. In addition, there is a need for a technique to determine whether a target nerve is effective in treating high blood pressure and to excise only the target nerve based on the determination results.

### DISCLOSURE OF THE INVENTION

### Technical Problem

Therefore, the present invention was created to solve the problems described above, and its purpose is to provide an invention, in which reaction of a target nerve is scanned and detected and only the target nerve is excised according to results of scanning and detecting.

In addition, the present invention aims to provide an invention, in which it is possible to detect whether a heating electrode that excises a target nerve is in contact with a blood vessel inner wall of a kidney.

However, the purposes of the present invention are not limited to those mentioned above, and other purposes not mentioned will be clearly understood by those skilled in the art from the description below.

### Technical Solution

The purposes of the present invention described above can be achieved by providing a renal denervation catheter, including a catheter housing, an electrode part, which is provided in the housing, emits a contact detection RF signal for contact with an inner wall of a renal vessel according to a contact detection mode, and emits a denervation RF signal for renal denervation according to a denervation mode, and a control unit for allowing entry into the denervation mode when a contact detection feedback signal value generated by the contact detection mode satisfies a defined condition.

In addition, the renal denervation catheter further includes a current detection unit for detecting a feedback current value generated by the contact detection RF signal.

In addition, the control unit includes a blood vessel inner wall contact check unit, which compares the feedback current value transmitted from the current detection unit with the pre-defined condition, and determines whether the electrode part is in contact with the inner wall of the renal vessel, and a mode switching unit, which switches and changes the contact detection mode and the denervation mode, and switches the contact detection mode to the denervation mode according to a control signal of the blood vessel inner wall contact check unit.

In addition, the renal denervation catheter further includes a frequency generating unit for generating an RF frequency corresponding to the mode switched by the mode switching unit and transmitting the RF frequency to the electrode part.

In addition, the frequency generating unit includes a blood vessel inner wall contact detection frequency generating unit for generating a blood vessel inner wall contact detection RF frequency according to the contact detection mode and transmitting the blood vessel inner wall contact detection RF frequency to the electrode part, and a denervation frequency generating unit for generating a denervation RF frequency according to the denervation mode and transmitting the denervation RF frequency to the electrode part.

Meanwhile, the purposes of the present invention can be achieved by providing a renal denervation catheter, including a catheter housing, an electrode part provided in the housing and emitting a nerve stimulation signal so as to detect reaction of a nerve to a target nerve area of a kidney, and a control unit for generating a scan/detection control signal to scan and detect whether the nerve responds to the nerve stimulation signal.

In addition, the nerve stimulation signal is a current pulse signal, in which positive and negative currents alternately repeat during one cycle, so that in a current pulse signal of a first cycle, positive current is generated first, followed by negative current, and in a current pulse signal of a second cycle, negative current is generated first, followed by positive current, and the current pulse signals of the first cycle and second cycle are periodically repeated.

In addition, the control unit includes a target scan mode unit, which generates the scan/detection control signal and changes position of the catheter and frequency or waveform of the nerve stimulation signal according to reaction sensitivity of a target nerve, and a denervation determination unit for determining whether to excise the target nerve by receiving a detection signal from the outside for reaction of the nerve in response to the nerve stimulation signal emitted by the electrode part.

In addition, the control unit further includes an excision position marking unit for marking an excision position of the target nerve according to target nerve excision determination of the denervation determination unit, and a denervation mode unit for generating a denervation control signal according to the target nerve excision determination of the denervation determination unit.

In addition, the renal denervation catheter includes a nerve stimulation frequency generating unit for generating the current pulse signal according to the control signal of the target scan mode unit and transmitting the control signal to the electrode part so as to enable detection of reaction of the target nerve, and a denervation frequency generating unit for generating the denervation RF frequency according to the control signal of the denervation mode unit and transmitting the denervation RF frequency to the electrode part so as to enable excision of the target nerve.

Meanwhile, the purposes of the present invention can be achieved by providing a renal denervation catheter, including a catheter housing, ring-shaped electrode parts, which are arranged in a ring type at preset intervals within a predetermined width in the circumferential direction of the housing and include heating electrodes for emitting a denervation RF signal so as to excise a target nerve of a kidney, a frequency generating unit for generating the denervation RF signal and transmits the denervation RF signal to the ring-shaped electrode parts, and a control unit for generating a control signal so as to enable generation of the denervation RF signal.

In addition, the ring-shaped electrode parts are divided into at least three sectors, and each of the sectors has a heating electrode disposed so as to excise the target nerve of the kidney.

In addition, a plurality of ring-shaped electrode parts are arranged at predetermined intervals in the longitudinal direction of the catheter housing.

In addition, the plurality of ring-shaped electrode parts are arranged so that positions of the heating electrodes disposed on each of the ring-shaped electrode parts are not the same as each other.

Meanwhile, the purposes of the present invention can be achieved by providing a renal denervation catheter, including a catheter housing, ring-shaped electrode parts, which are arranged in a ring type at preset intervals within a predetermined width in the circumferential direction of the housing, and include detection electrodes for emitting a nerve stimulation signal so as to detect reaction of a nerve in a target nerve area of a kidney and heating electrodes for emitting a denervation RF signal so as to excise a target nerve of a kidney, and a control unit for generating a control signal so as to enable generation of the nerve stimulation signal and the denervation RF signal.

In addition, the ring-shaped electrode parts are arranged, so that the detection electrodes and the heating electrodes are alternately repeated, and are divided into at least three sectors, respectively.

In addition, the control unit includes a target scan mode unit, which generates a scan/detection control signal and changes position of the catheter and frequency of the nerve stimulation signal according to reaction sensitivity of a target nerve, a target area determination unit for determining an excision area of the target nerve by receiving a detection signal from the outside for reaction of the nerve in response to the nerve stimulation signal emitted by the detection electrode, a denervation mode unit for generating a denervation control signal according to excision area determination of the target area determination unit, and a mode setting unit for controlling mode switching of the target scan mode unit and the denervation mode unit.

In addition, the target scan mode unit includes a first target scan mode unit for allowing a pair of first and second detection electrodes to emit first and second nerve stimulation signals to the target nerve and to detect reaction of a nerve adjacent to a first heating electrode disposed between the first and second detection electrodes, a second target scan mode unit for allowing a pair of second and third detection electrodes to emit first and second nerve stimulation signals to a target nerve and to detect reaction of the nerve adjacent to a second heating electrode disposed between the second and third detection electrodes, and a third target scan mode unit for allowing a pair of first and third detection electrodes to emit first and second nerve stimulation signals to a target nerve and to detect reaction of the nerve adjacent to a third heating electrode disposed between the first and third detection electrodes.

In addition, each of the first and second nerve stimulation signals is a current pulse signal, in which positive and negative currents alternately repeat during one cycle, so that in a current pulse signal of a first cycle, positive current is generated first, followed by negative current, and in a current pulse signal of a second cycle, negative current is generated first, followed by positive current, and the current pulse signals of the first cycle and second cycle are periodically repeated.

In addition, the second nerve stimulation signal is emitted to the target nerve in a discharge section of the first nerve stimulation signal.

In addition, a plurality of ring-shaped electrode parts are arranged at predetermined intervals in the longitudinal direction of the catheter housing.

In addition, the plurality of ring-shaped electrode parts are arranged so that positions of the heating electrodes and the detection electrodes disposed on each of the ring-shaped electrode parts are not the same as each other.

In addition, the mode setting unit includes a detection/excision/fixation mode unit for detecting and excising the target nerve while fixing the ring-shaped electrode part, and a detection/excision/movement mode unit for detecting and excising the target nerve while moving the ring-shaped electrode part along a renal vessel, so as to disperse a nerve excision area.

In addition, the renal denervation catheter includes a nerve stimulation frequency generating unit for generating the current pulse signal according to the control signal of the target scan mode unit and transmitting the control signal to the detection electrode so as to enable detection of reaction of the target nerve, and a denervation frequency generating unit for generating the denervation RF frequency according to the control signal of the denervation mode unit and transmitting the denervation RF frequency to the heating electrode so as to enable excision of the target nerve.

### Advantageous Effects of the Invention

According to the present invention as described above, reaction of a target nerve is scanned and detected, and only the target nerve is excised according to the scanning and detection result.

In addition, according to the present invention, it is possible to efficiently excise a target nerve by detecting whether a heating electrode that excises the target nerve is in contact with a blood vessel inner wall of a kidney.

### BRIEF DESCRIPTION OF DRAWINGS

The following drawings attached herein illustrate preferred embodiments of the present invention and serve to further understand the technical idea of the present invention along with a detailed description of the invention, so the present invention should not be construed as limited to the matters illustrated in such drawings.

FIG. 1 is a diagram showing a catheter according to a first embodiment of the present invention,

FIG. 2 is a diagram schematically showing configuration of the catheter according to the first embodiment of the present invention,

FIG. 3 is a diagram showing a catheter according to a second embodiment of the present invention,

FIG. 4 is a diagram schematically showing configuration of the catheter according to the second embodiment of the present invention,

FIG. 5 is a diagram showing an example of a nerve stimulation signal according to the second embodiment of the present invention,

FIG. 6 is a diagram showing configuration of a catheter according to a third embodiment of the present invention,

FIGS. 7(a)-7(c) are diagrams showing first, second and third ring-shaped heating electrode parts according to the third embodiment of the present invention,

FIG. 8 is a diagram showing the first ring-shaped electrode part according to a fourth embodiment of the present invention,

FIG. 9, FIG. 11 and FIG. 13 show diagrams of schematic configurations of a catheter for detecting reaction sensitivity of each target nerve adjacent to each blood vessel inner wall C1, C2, C3 according to the fourth embodiment of the present invention,

FIG. 10, FIG. 12 and FIG. 14 show diagrams of detection electrode signals for detecting reaction sensitivity of each target nerve adjacent to each blood vessel inner wall C1, C2, C3 according to the fourth embodiment of the present invention,

FIG. 15 is a diagram showing that detection electrodes and heating electrodes according to the fourth embodiment of the present invention, wherein the detection electrodes and the heating electrodes are arranged so that their positions and directions do not overlap each other, and

FIG. 16 is a diagram showing schematic configuration of the catheter according to the fourth embodiment of the present invention.

### MODE FOR INVENTION

Hereinafter, preferred embodiments of the present invention will be described with reference to the accompanying drawings. In addition, the embodiments described below should not be construed as unduly limiting the content of the present invention described in the claims, and the entire configuration described in the embodiments cannot be interpreted as essential as a solution of the present invention. Furthermore, descriptions of prior art or matters that are obvious to those skilled in the art may be omitted, and descriptions of these omitted components (methods) and functions may be sufficiently referenced without departing from the technical idea of the present invention.

A renal denervation catheter according to an embodiment of the present invention is a device for treating high blood pressure by excising (ablation) or removing (denervation) nerves around kidney arteries or blood vessels. Hereinafter, a renal denervation catheter according to each embodiment will be described in detail with reference to the accompanied drawings.

### First Embodiment

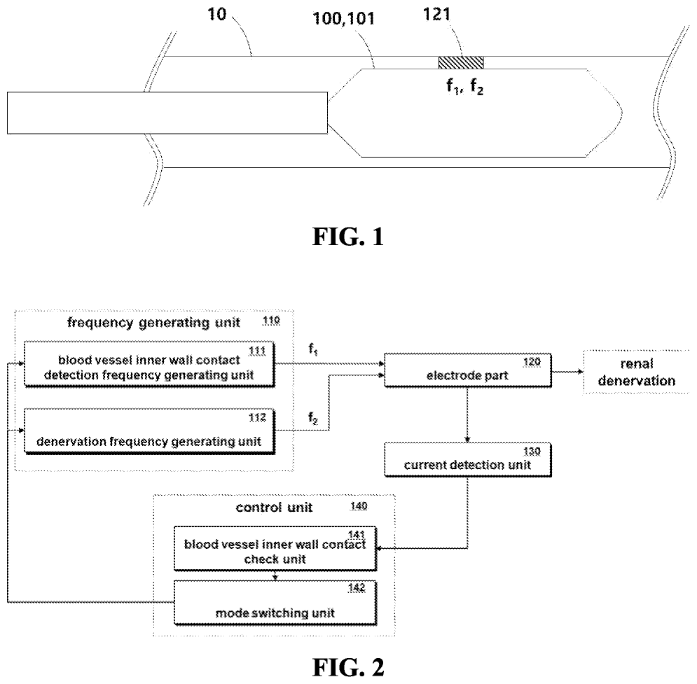

A renal denervation catheter according to a first embodiment of the present invention includes, as shown in FIG. 1 and FIG. 2, a catheter housing 101, a frequency generating unit 110, an electrode part 120, a current detection unit 130, and a control unit 140.

The catheter housing 101 may be made of a non-conductive or conductive material and, after reaching a target area, may be inflated like a balloon for nerve excision or removal (hereinafter, referred to as excision) as necessary. Prior art related to the catheter housing 101 adopted in a catheter for excision of a renal nerve may be referred to without departing from the technical spirit of the present invention.

The frequency generating unit 110 generates an RF frequency corresponding to a contact detection mode or denervation mode switched by a mode switching unit 142 and transmits the RF frequency to the electrode part 120. To this end, the frequency generating unit 110 includes a blood vessel inner wall contact detection frequency generating unit 111 or a denervation frequency generating unit 112.

The blood vessel inner wall contact detection frequency generating unit 111 generates a blood vessel inner wall contact detection RF frequency in the contact detection mode and transmits the blood vessel inner wall contact detection RF frequency to the electrode part 120. That is, when the catheter housing 101 reaches a target area of a renal vessel 10 (or a renal artery), the catheter housing 101 emits an RF signal through the electrode part 120 so as to excise a nerve around the renal vessel. Herein, for effective excision, it is preferable that the electrode part 120 is in close contact with an inner wall of the renal vessel. Therefore, the blood vessel inner wall contact detection frequency generating unit 111 generates an RF frequency f1 for detecting whether the electrode part 120 is in close contact with the inner wall of the renal vessel and transmits the RF frequency f1 to the electrode part 120. The contact detection RF frequency may be a current pulse signal as shown in FIG. 5. The contact detection RF is not limited to FIG. 5 in terms of frequency and waveform format and may vary depending on the environment.

The denervation frequency generating unit 112 generates a denervation RF frequency f2 in the denervation mode and transmits the denervation RF frequency f2 to the electrode part 120. Waveform and frequency of the denervation RF frequency can be selected in order to excise a nerve positioned in a surrounding area of a renal vessel. The electrode part 120 converts the transmitted denervation RF frequency into predefined RF power and emits the RF power to the nerve around the renal vessel.

The electrode part 120 is configured to include electrodes on the outside of the housing 101, and it is preferable that a plurality of electrodes are provided at different positions as needed. The electrode part 120 emits a contact detection RF signal for contact with an inner wall of a renal vessel according to the contact detection mode, and emits a denervation RF signal for renal denervation according to the denervation mode. Herein, the electrode part 120 may separately include a heating electrode part (not shown) including a heating electrode and a contact detection electrode part (not shown) including a contact detection electrode. When the contact detection electrode part is configured separately, the current detection unit 130 detects feedback current of an RF signal emitted by the contact detection electrode part. The contact detection electrode and the heating electrode are disposed on a surface of the housing 101. However, the contact detection electrode is an electrode for detecting whether the heating electrode is in contact with a surface of a renal vessel, and is preferably placed adjacent to the heating electrode. That is, when the heating electrode and the contact detection electrode are configured separately, it is preferable to arrange the heating electrode and the contact detection electrode in pairs on the surface of the housing 101.

The control unit 140 controls operation of the catheter on the whole and includes a blood vessel inner wall contact check unit 141 and a mode switching unit 142. The blood vessel inner wall contact check unit 141 compares a feedback current value transmitted from the current detection unit 130 with a pre-defined condition, and if the condition is satisfied, it is determined that the heating electrode part is in contact with an inner wall of a renal vessel, so that a control signal according to the satisfaction of the condition is transmitted to the mode switching unit 142 so as to carry out entry into the denervation mode.

The mode switching unit 142 switches the contact detection mode and the denervation mode, and switches the contact detection mode to the denervation mode according to the control signal of the blood vessel inner wall contact check unit 141.

As an example, when the catheter according to the first embodiment of the present invention enters a target area, the mode switching unit 142 first enters the contact detection mode. Upon entering the contact detection mode, the blood vessel inner wall contact detection frequency generating unit 111 generates a blood vessel inner wall contact detection RF frequency. The generated blood vessel inner wall contact detection RF frequency is emitted to the target area (to a blood vessel inner wall) by the contact detection electrode part. The current detection unit 130 detects feedback current generated by the emitted blood vessel inner wall contact detection RF frequency, and the detected feedback current is transmitted to the blood vessel inner wall contact check unit 141. The blood vessel inner wall contact check unit 141 determines whether the heating electrode part is in contact with the blood vessel inner wall of a kidney.

When contact with the blood vessel inner wall is determined, the mode switching unit 142 enters the denervation mode, so that upon entering the denervation mode, the denervation frequency generating unit 112 generates a denervation RF frequency. The generated denervation RF frequency is emitted to the target area (to a nerve around the renal vessel) by the heating electrode part so that the nerve is excised.

### Second Embodiment

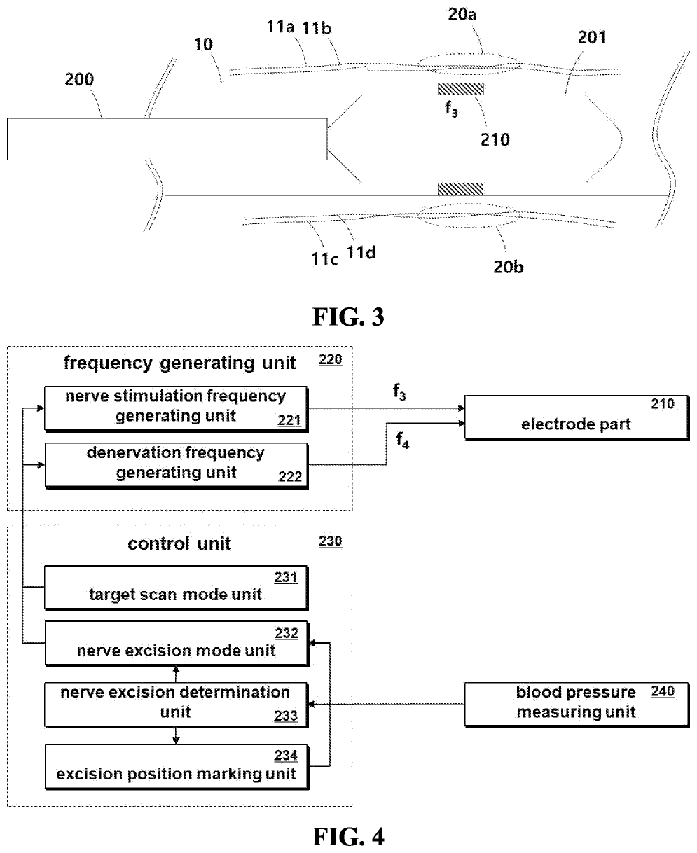

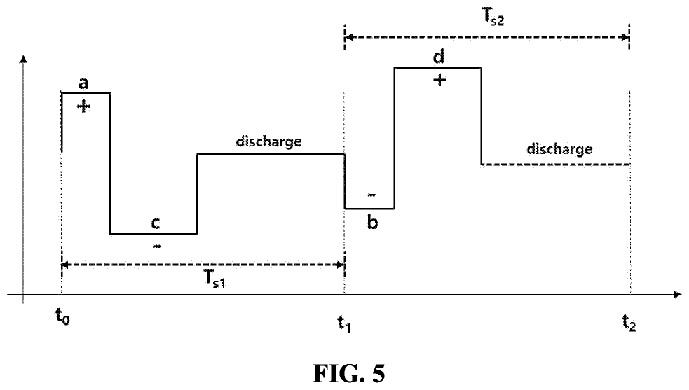

A renal denervation catheter according to a second embodiment of the present invention includes, as shown in FIG. 3 to FIG. 5, a catheter housing 201, an electrode part 210, a frequency generating unit 220 and a control unit 140. The description of the catheter housing 201 will be replaced with that of the first embodiment.

The electrode part 210 is provided on a surface of the housing 201 and emits a nerve stimulation signal to detect reaction of a nerve in a target nerve area of a kidney. In other words, it is intended to detect whether reaction of a nerve is inhibited by emitting a nerve stimulation signal to a target nerve. When the nerve responds to the nerve stimulation signal, blood pressure is lowered so that the target nerve requiring excision can be discovered.

The electrode part 210 may separately or integrally include a detection electrode part (not shown) provided with a detection electrode and a heating electrode part (not shown) provided with a heating electrode. In the case of integrated configuration, only a frequency input for each function may be different. The detection electrode part is intended to detect whether the target nerve responses, and the heating electrode part is intended to excise the target nerve detected by the detection electrode part. The placement and numbers of the detection electrode and the heating electrode can be appropriately adopted as needed.

The frequency generating unit 220 includes a nerve stimulation frequency generating unit 221 and a denervation frequency generating unit 222.

The nerve stimulation frequency generating unit 221 generates a current pulse signal f3 according to a control signal of a target scan mode unit 231 and transmits the current pulse signal f3 to the electrode part 210 (the detection electrode part), thereby detecting reaction of nerves 11a, 11b, 11 c, 11d. Frequency or waveform of the current pulse signal can be selectively changed according to reaction sensitivity of target nerves 20a, 20b.

FIG. 5 shows an example of a nerve stimulation signal, wherein the nerve stimulation signal is a current pulse signal, in which positive and negative currents alternately repeat during one cycle.

In a current pulse signal of a first cycle Ts1, positive current (area “a”) is generated first, followed by negative current (area “c”), and in a current pulse signal of a second cycle Ts2, negative current (area “b”) is generated first, followed by positive current (area “d”). The current pulse signals of the first cycle Ts1 and the second cycle Ts2 repeats periodically. Herein, it is preferable that width of “a” is the same as width of “b”, and width of “c” is the same as width of “d”. The size, frequency (for example, Hz, Khz), waveform (for example, square wave or triangle wave), etc. of the positive current and the negative current in each area can be selectively changed depending on the reaction sensitivity of the target nerve.

The denervation frequency generating unit 222 generates a denervation RF frequency f4 according to a control signal of a denervation mode unit and transmits the denervation RF frequency f4 to the electrode part 210 (the heating electrode part) to excise the target nerve.

The control unit 230 generates a scan/detection control signal to scan and detect whether the nerve responds to the nerve stimulation signal and a denervation control signal to excise the target nerve if the nerve responds according to the scanning and detection. To this end, the control unit 230 includes a target scan mode unit 231, a nerve excision mode unit 232, a nerve excision determination unit 233, and an excision position marking unit 234.

The target scan mode unit 231 generates the scan/detection control signal and changes position of the catheter or frequency or waveform of the nerve stimulation signal, depending on reaction sensitivity of the target nerve. If the target nerves 20a, 20b do not respond, it is possible to change the position or direction of the catheter so as to carry out detection again or to newly scan and detect adjacent target nerves. Additionally, the frequency or waveform of the nerve stimulation signal can be selectively changed depending on reaction sensitivity of the target nerves.

The denervation determination unit 233 determines whether to excise the target nerves by receiving from an external blood pressure measuring unit 240 a detection signal for the response of the target nerves according to the nerve stimulation signal emitted by the electrode part 210 (the detection electrode part). In other words, when the target nerves respond to the nerve stimulation signal, the blood pressure of an examinee or a subject who is treated (hereinafter, referred to as “subject”) changes (preferably, the blood pressure decreases), and accordingly, the blood pressure measuring unit 240 can detect such a change in the subject's blood pressure. A blood pressure value measured by the blood pressure measuring unit 240 is transmitted to the denervation determination unit 233, and the denervation determination unit 233 determines whether a reaction has occurred by comparing the blood pressure measured after reaction with baseline blood pressure of the subject and a pre-defined condition.

Herein, the denervation determination unit 233 determines not to excise the detected target nerves if the pre-defined condition is not satisfied because there is no change or a slight change in the subject's blood pressure. In other words, sensitivity to respond to nerve stimulation signals may vary from subject to subject, and furthermore, there may be subjects who do not respond to nerve stimulation signals. Therefore, there is an advantage in that a target nerve is excised according to reaction sensitivity of a subject rather than unconditionally excising the target nerve. Of course, the denervation determination unit 233 determines to excise the detected target nerves when a change occurs in the subject's blood pressure and the predefined condition is satisfied.

The excision position marking unit 234 marks an excision position of the target nerve according to the target nerve excision determination of the denervation determination unit 233. The excision position marking unit 234 can mark the position of the detected target nerve with a visible color or by lightly burning a blood vessel around the target nerve. As another example, the excision position marking unit 234 may implant a chip that can be removed after a procedure and can transmit a position around the excision position of the target nerve. The excision position marking unit 234 informs an operator of the position of the target nerve and can be implemented by various methods.

The denervation mode unit 232 generates the denervation control signal according to the target nerve excision determination of the denervation determination unit 233. According to the denervation control signal, the denervation frequency generating unit 222 generates a denervation RF signal and transmits the denervation RF signal to the heating electrode.

As an example, when the catheter according to the second embodiment of the present invention enters a target area, the target scan mode unit 231 allows entry into a scan/detection mode and transmits a control signal to the nerve stimulation frequency generating unit 221 so that a nerve stimulation signal is generated upon entry into the scan/detection mode. The nerve stimulation frequency generating unit 221 transmits the generated nerve stimulation signal to the detection electrode part, and the detection electrode part emits the nerve stimulation signal to a nerve in the target area. According to the emission of the nerve stimulation signal, the blood pressure measuring unit 240 measures a blood pressure change of the subject and transmits the blood pressure change to the nerve excision determination unit 233.

If there is no change in the subject's blood pressure, the nerve excision determination unit 233 can generate a control signal and transmits the control signal to the target scan mode unit 231 so as to change the detection target area, or may terminate operation without performing nerve excision after target nerve detection depending on nerve reaction sensitivity of the subject. If there is a change in the subject's blood pressure, the nerve excision determination unit 233 generates a control signal and transmits the control signal to the excision position marking unit 234 so as to mark the detected target area, and transmits a control signal to the nerve excision mode unit 232 so as to allow excision of the target nerve around the marked target.

The nerve excision mode unit 232 allows entry into the denervation mode according to the control signal of the nerve excision determination unit 233, and transmits a control signal to the denervation frequency generating unit 222 so that a denervation RF frequency is generated according to a denervation mode. The denervation frequency generating unit 222 transmits the generated denervation RF frequency to the heating electrode part so that the target nerve is excised.

### Third Embodiment

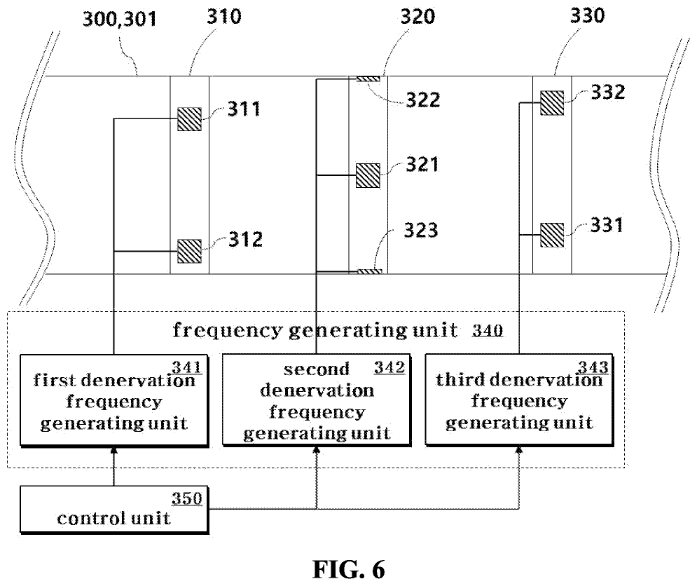

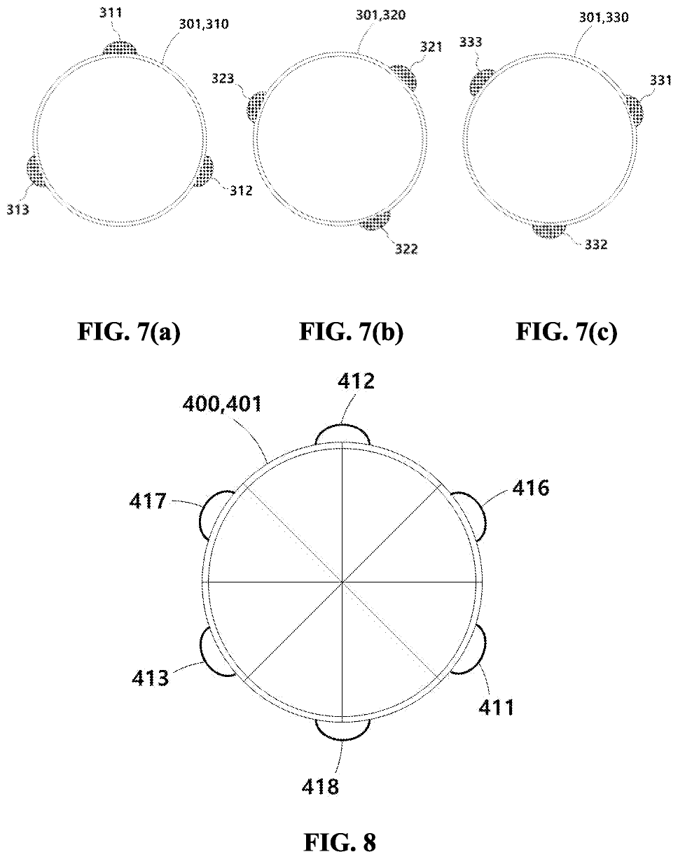

A renal denervation catheter according to a third embodiment of the present invention includes, as shown in FIG. 6 and FIGS. 7(a)-7(c), a catheter housing 301, first, second and third ring-shaped heating electrode parts 310, 320, 330, a frequency generating unit 340 and a control unit 350. The description of the catheter housing 301 will be replaced with that of the first embodiment.

The first ring-shaped heating electrode part 310 is arranged in a ring type at a preset interval within a predetermined width in the circumferential direction of the housing 301 and includes first heating electrodes 311, 312, 313 for emitting a denervation RF signal to excise a target nerve of a kidney. Like the first ring-shaped heating electrode part 310, the second ring-shaped heating electrode part 320 is provided with second heating electrodes 321, 322, 323, and the third ring-shaped heating electrode part 330 includes third heating electrodes 331, 332, 333. It is preferable that each of the first, second and third ring-shaped heating electrode parts 310, 320, 330 are arranged so that positions or directions of the heating electrodes placed in each ring-shaped electrode part are not the same (or do not overlap), as shown in FIGS. 6 and FIGS. 7(a)-7(c). The first, second and third ring-shaped heating electrode parts 310, 320, 330 are arranged in plural numbers at predetermined intervals in the longitudinal direction of the catheter housing 301. The number of the electrode parts can be selectively adopted depending on length of a blood vessel.

The ring type means that the first heating electrodes 311, 312, 313 arranged in the first ring-shaped heating electrode part 310 are arranged at a uniform distance and within a predetermined width in the circumferential direction of a surface of the housing 301. Therefore, the second and third ring-shaped heating electrode parts 320, 330 are also arranged in the same ring type. As an example, if three heating electrodes are placed at a uniform distance in the circumferential direction of the housing 301 as shown in FIG. 7(a), each heating electrode can be placed in an area of three divided sectors of 120 degrees. Of course, the heating electrodes can be placed by dividing the sectors evenly according to the number of heating electrodes. It is preferable that the first, second and third heating electrodes 311, 312, 313, 321, 322, 323, 331, 332, 333 are arranged so that positions or directions of each heating electrode are not the same (or do not overlap) as each other as shown in FIG. 7(a)-(c).

The frequency generating unit 340 generates a denervation RF frequency and transmits the denervation RF frequency to each heating electrode of the first, second and third ring-shaped heating electrode parts 310, 320, 330. Herein, the frequency generating unit 340 may include first, second and third denervation frequency generating units 341, 342, 343 for transmitting a denervation RF frequency to the first, second and third ring-shaped heating electrode parts 310, 320, 330, respectively. In other words, when denervation RF signals are distributed and transmitted to each of the first, second and third ring-shaped heating electrode parts 310, 320, 330 by one frequency generating unit 340, it is not possible to independently control frequency or waveform of the first, second and third ring-shaped heating electrode parts 310, 320, 330 or independently turn on/off the first, second and third ring-shaped heating electrode parts 310, 320, 330. Therefore, it may be preferable to include the first, second and third denervation frequency generating units 341, 342, 343.

The control unit 350 can control the frequency, size, waveform, etc. of the denervation RF signals generated by the first, second and third denervation frequency generating units 341, 342, 343. Operation of any one denervation frequency generating unit can be turned off as necessary. A heating electrode electrically connected to such an off denervation frequency generating unit does not emit a denervation RF signal. Therefore, the emission of the denervation RF signal can be controlled locally.

Additionally, the control unit 350 can individually adjust the size of the RF power of the denervation RF frequency emitted by each heating electrode of the first, second and third ring-shaped heating electrode parts 310, 320, 330.

### Fourth Embodiment

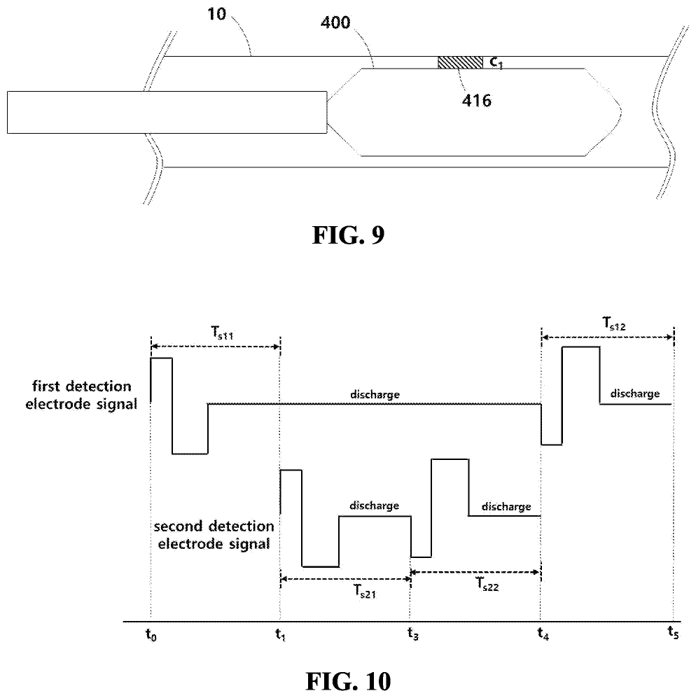

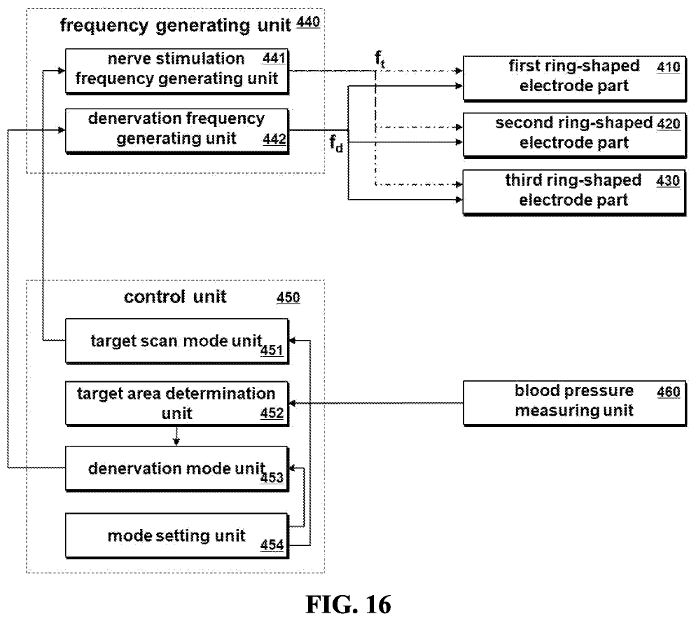

A renal denervation catheter according to a fourth embodiment of the present invention includes, as shown in FIG. 8 to FIG. 16, a catheter housing 401, first, second and third ring-shaped electrode part 410, 420, 430, a frequency generating unit 440, a control unit 450, and a blood pressure measuring unit 460. The description of the catheter housing 401 will be replaced with that of the first embodiment.

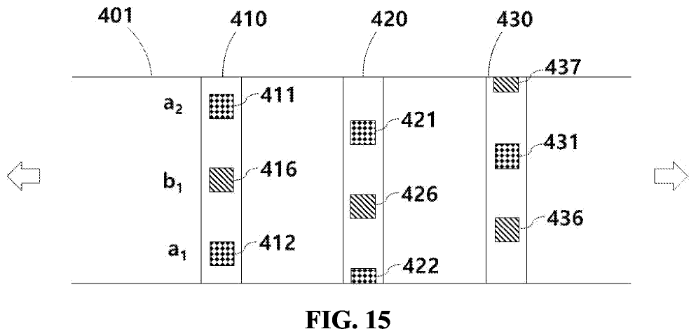

The first, second and third ring-shaped electrode parts 410, 420, 430 are arranged in a ring type at preset intervals within a predetermined width in the circumferential direction of the housing 401, and respectively include a detection electrode which emits a nerve stimulation signal so as to detect reaction of a nerve in a target nerve area of a kidney and a heating electrode which emits a denervation RF signal so as to excise a target nerve of the kidney. As an example, as shown in FIG. 8, the first ring-shaped electrode part 410 is provided with first, second and third detection electrodes 411, 412, 413 and first, second and third heating electrodes 416, 417, 418. The first, second and third detection electrodes 411, 412, 413 are placed in three sectors divided by 120 degrees so as to be spaced apart from each other by 120 degrees, and the first, second and third heating electrodes 416, 417, 418 are also placed at 120 degrees apart from each other in the same way. Accordingly, a spacing distance between the detection electrodes and a spacing distance between the heating electrodes are the same as each other, and the detection electrodes and the heating electrodes are arranged alternately each other. The second and third ring-shaped electrode parts 420, 430 are also arranged in the same manner. The detection electrodes and the heating electrodes of the first, second and third ring-shaped electrode parts 410, 420, 430 may be respectively arranged differently from each other in position or direction depending on a treatment mode, as shown in FIG. 15. A plurality of first, second and third ring-shaped electrode parts 410, 420, 430 are arranged at predetermined intervals in the longitudinal direction of the catheter housing 401. It is possible to dispose a plurality of ring-shaped electrode parts or a single ring-shaped electrode part as necessary.

The control unit 450 generates a control signal to generate a nerve stimulation signal and a denervation RF signal. Herein, the control unit 450 can selectively and independently control the first, second and third ring-shaped electrode parts 410, 420, 430. Selective control means controlling by stopping operation of at least one of the first, second and third ring-shaped electrode parts 410, 420, 430, and independent control means independently controlling the frequency, waveform, signal size, etc. supplied to the first, second and third ring-shaped electrode parts 410, 420, 430.

The control unit 450 includes a target scan mode unit 451, a target area determination unit 452, a denervation mode unit 453, and a mode setting unit 454.

The target scan mode unit 451 generates a scan/detection control signal and changes position of the catheter and frequency or waveform of the nerve stimulation signal depending on reaction sensitivity of the target nerve. In other words, when reaction of the target nerve is minimal or absent, it is possible to change frequency of the nerve stimulation signal, or waveform or signal size thereof according to determination result of the target area determination unit 452.

The target scan mode unit 451 includes a first target scan mode unit (not shown), a second target scan mode unit (not shown), and a third target scan mode (not shown). As an example, description is provided based on the first ring-shaped electrode part 410, but the second and third ring-shaped electrode parts 420, 430 can also be operated in the same manner.

The first target scan mode unit allows a pair of first and second detection electrodes 411, 412 to emit first and second nerve stimulation signals to the target nerve and to detect reaction of the target nerve positioned in the vicinity of a blood vessel inner wall C1 adjacent to the first heating electrode 416 disposed between the first and second detection electrodes 411, 412.

The second target scan mode unit allows a pair of second and third detection electrodes 412, 413 to emit first and second nerve stimulation signals to a target nerve and to detect reaction of the target nerve positioned in the vicinity of a blood vessel inner wall C2 adjacent to the second heating electrode 417 disposed between the second and third detection electrodes 412, 413.

The third target scan mode unit allows a pair of first and third detection electrodes 411, 413 to emit first and second nerve stimulation signals to a target nerve and to detect reaction of the target nerve positioned in the vicinity of a blood vessel inner wall C3 adjacent to the third heating electrode 418 disposed between the first and third detection electrodes 411, 413.

As described above, if detection is carried out by pairing detection electrodes arranged in directions different from each other, the most reliable nerve response can be detected in each direction, wherein the control unit 450 can selectively and independently control the first, second and third heating electrodes 416, 417, 418 of the first ring-shaped electrode part 410 so as to excise a most reliable target nerve. As an example, when reaction of the target nerve detected by the third target scan mode unit is the most reliable, the third heating electrode 418 can be operated selectively and independently without operating the first and second heating electrodes 416, 417.

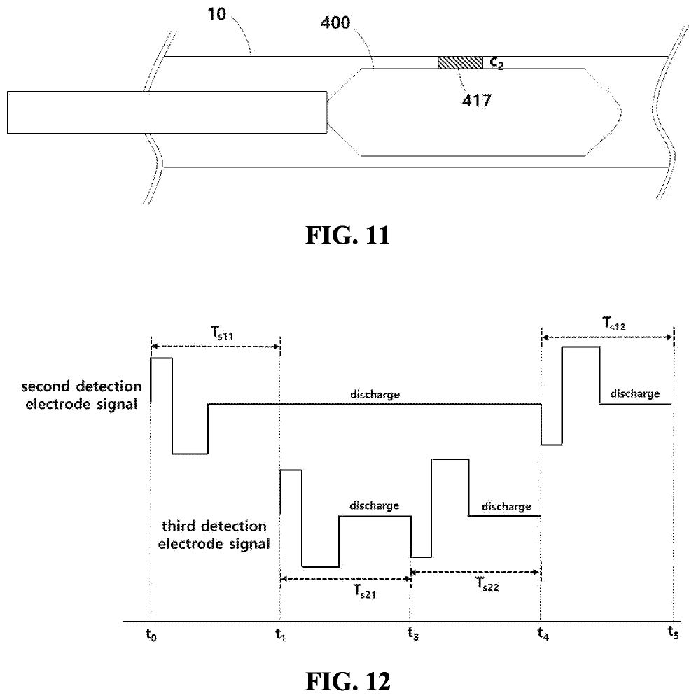

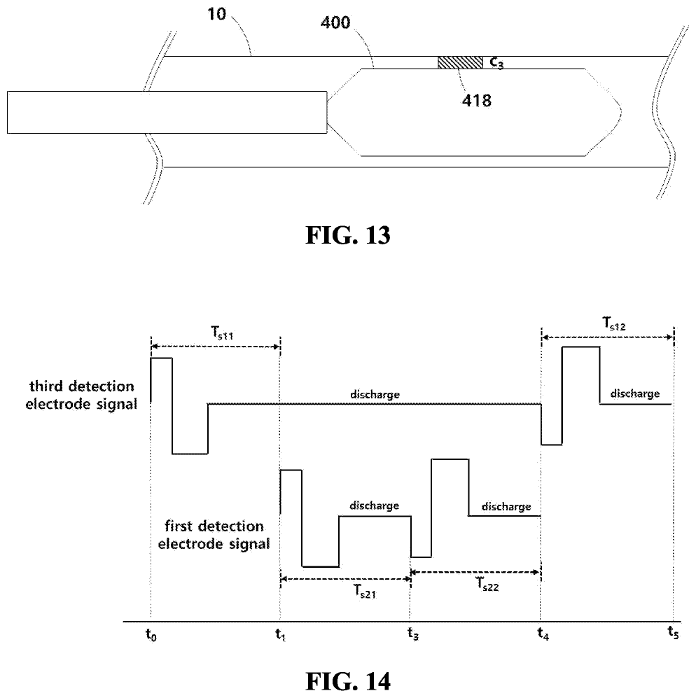

The first and second nerve stimulation signals described above are respectively current pulse signals in which positive current and negative current alternately repeat during one cycle Ts11, Ts12, Ts21, Ts22, as shown in FIG. 10, FIG. 12 and FIG. 14. As an example, in a current pulse signal of a first cycle Ts11, positive current is generated first, followed by negative current, and in a current pulse signal of a second cycle Ts12, negative current is generated first, followed by positive current. In addition, the current pulse signals of the first cycle Ts11 and the second cycle Ts12 are periodically repeated. Description of these current pulse signals will be replaced with that of the current pulse signal shown in FIG. 5.

The second nerve stimulation signal is generated to be emitted to the target nerve within a discharge section of the first nerve stimulation signal, as shown in FIG. 10, FIG. 12 and FIG. 14.

Meanwhile, referring to FIG. 10, the first nerve stimulation signal of the first target scan mode unit is a first detection electrode signal emitted from the first detection electrode 411, and the second nerve stimulation signal is a second detection electrode signal emitted from the second detection electrode 412.

Referring to FIG. 12, the first nerve stimulation signal of the second target scan mode unit is the second detection electrode signal emitted from the second detection electrode 412, and the second nerve stimulation signal is a third detection electrode signal emitted from the third detection electrode 413.

Referring to FIG. 14, the first nerve stimulation signal of the third target scan mode unit is the third detection electrode signal emitted from the third detection electrode 413, and the second nerve stimulation signal is the first detection electrode signal emitted from the first detection electrode 411.

The target area determination unit 452 determines an excision area of a target nerve by receiving from the blood pressure measuring unit 460 a detection signal for reaction of a nerve according to a nerve stimulation signal emitted from a detection electrode. In other words, through the emission of the nerve stimulation signals according to the first, second and third target scan mode units as described hereinabove, the most reliable reaction among the reactions of the target nerves in each mode is detected and recognized, and accordingly a control signal is transmitted to the nerve excision mode unit 453 so that only the nerve associated with the most reliable reaction is targeted and excised.

The denervation mode unit 453 generates a denervation control signal according to determination of a nerve excision site by the target area determination unit 452. As an example, the denervation mode unit 453 does not operate all the first, second and third heating electrodes 416, 417, 417 of the first ring-shaped electrode part 410, but selectively and independently operates a heating electrode positioned between a pair of detection electrodes that shows most reliable reaction according to detection results of the first, second and third target scan mode units as described above, so that a target nerve is excised.

The mode setting unit 454 controls mode switching of the target scan mode unit and the denervation mode unit. In addition, the mode setting unit 454 controls mode switching of a detection/excision/fixation mode unit and a detection/excision/movement mode unit.

The detection/excision/fixation mode unit detects and excises a target nerve as described above while fixing the ring-shaped electrode parts. The detection/excision/movement mode unit disperses a nerve excision area by detecting and excising target nerves while moving the ring-shaped electrode parts along a renal vessel. The detection/excision/movement mode unit repeats the process of detecting and excising a first spot in a detection area, then moving along the longitudinal direction of the renal vessel and detecting and excising a second spot. In the detection/excision/movement mode unit, it is preferable that the detection electrodes and heating electrodes placed in the first, second and third ring-shaped electrode parts 410, 420, 430 are not disposed at same positions or in same directions so that they do not overlap each other. Meanwhile, depending on a situation at each spot, only detection may be performed and excision may not be performed depending on reaction sensitivity.

The frequency generating unit 440 includes a nerve stimulation frequency generating unit 441 and a denervation frequency generating unit 442. The nerve stimulation frequency generating unit 441 generates a current pulse signal and transmitting the current pulse signal to each detection electrode according to a control signal of the target scan mode unit 451, so that reaction of a target nerve is detected. Herein, the nerve stimulation frequency generating unit 441 may be individually configured to selectively and independently transmit nerve stimulation frequency to each detection electrode provided in the first, second and third ring-shaped electrode parts 410, 420, 430.

The denervation frequency generating unit 442 generates denervation RF frequency according to a control signal of the denervation mode unit 453 and transmits the denervation RF frequency to each heating electrode so that a target nerve is excised. Herein, the denervation frequency generating unit 442 may be individually configured to selectively and independently transmit RF frequency to each heating electrode provided in the first, second and third ring-shaped electrode parts 410, 420, 430.

In describing the present invention, prior art or matters that would be obvious to those skilled in the art to which the present invention belongs may be omitted, and descriptions of these omitted components (methods) and functions could be sufficiently referenced without departing from the technical idea of the present invention. In addition, the components of the present invention described above are merely explained for the convenience of explaining the present invention, and components not described herein may be added without departing from the technical spirit of the present invention.

The description of the composition and function of each part mentioned above is presented separately only for convenience of explanation, and any one of such composition and function may be implemented integrated with other components or may be further subdivided as needed.

Hereinabove, the present invention has been described with reference to embodiments, but the present invention is not limited thereto, and various modifications and applications can be made. In other words, those skilled in the art would readily understand that many modifications can be made without departing from the gist of the present invention. In addition, it is noted that if it is judged that specific explanation on well-known function and components related to the present invention or the coupling relationship of each component of the present invention may unnecessarily obscure the gist of the present invention, the detailed explanation has been omitted.

### EXPLANATION OF REFERENCE SIGNS

10: renal artery or renal vessel
11, 11a, 11b, 11c, 11d: nerve
20a, 20b: target nerve
100: catheter
101: housing
110: frequency generating unit
111: blood vessel inner wall contact detection frequency generating unit
112: denervation frequency generating unit
120: electrode part
121: heating electrode
130: current detection unit
140: control unit
141: blood vessel inner wall contact check unit
142: mode switching unit
200: catheter
201: housing
210: electrode part
220: frequency generating unit
221: nerve stimulation frequency generating unit
222: denervation frequency generating unit
230: control unit
231: target scan mode unit
232: denervation mode unit
233: denervation determination unit
234: excision position marking unit
240: blood pressure measuring unit
300: catheter
301: housing
310: first ring-shaped heating electrode part
311, 312, 313: first heating electrode
320: second ring-shaped heating electrode part
321, 322, 323: second heating electrode
330: third ring-shaped heating electrode part
331, 332, 333: third heating electrode
340: frequency generating unit
341: first denervation frequency generating unit
342: second denervation frequency generating unit
343: third denervation frequency generating unit
350: control unit
400: catheter
401: housing
410: first ring-shaped electrode part
411, 412, 413: first, second and third detection electrode
416, 417, 418: first, second and third heating electrode
420: second ring-shaped electrode part
421, 422: first and second detection electrode
426: first heating electrode
430: third ring-shaped electrode part
431: first detection electrode
436, 437: first and second heating electrode
440: frequency generating unit
441: nerve stimulation frequency generating unit
442: denervation frequency generating unit
450: control unit
451: target scan mode unit or target detection mode unit
452: target area determination unit
453: nerve excision mode unit
454: mode setting unit
460: blood pressure measuring unit

1. A renal denervation catheter, comprising:
a catheter housing;
an electrode part, which is provided in the housing, emits a contact detection RF signal for contact with an inner wall of a renal vessel according to a contact detection mode, and emits a denervation RF signal for renal denervation according to a denervation mode; and
a control unit for allowing entry into the denervation mode when a contact detection feedback signal value generated by the contact detection mode satisfies a defined condition.

2. The catheter according to claim 1, further comprising a current detection unit for detecting a feedback current value generated by the contact detection RF signal.

3. The catheter according to claim 2, wherein
the control unit includes:
a blood vessel inner wall contact check unit, which compares the feedback current value transmitted from the current detection unit with the pre-defined condition, and determines whether the electrode part is in contact with the inner wall of the renal vessel; and
a mode switching unit, which switches and changes the contact detection mode and the denervation mode, and switches the contact detection mode to the denervation mode according to a control signal of the blood vessel inner wall contact check unit.

4. The catheter according to claim 3, further comprising a frequency generating unit for generating an RF frequency corresponding to the mode switched by the mode switching unit and transmitting the RF frequency to the electrode part.

5. The catheter according to claim 4, wherein
the frequency generating unit includes:
a blood vessel inner wall contact detection frequency generating unit for generating a blood vessel inner wall contact detection RF frequency according to the contact detection mode and transmitting the blood vessel inner wall contact detection RF frequency to the electrode part; and
a denervation frequency generating unit for generating a denervation RF frequency according to the denervation mode and transmitting the denervation RF frequency to the electrode part.

6. A renal denervation catheter, comprising:
a catheter housing;
an electrode part provided in the housing and emitting a nerve stimulation signal so as to detect reaction of a nerve to a target nerve area of a kidney; and
a control unit for generating a scan/detection control signal to scan and detect whether the nerve responds to the nerve stimulation signal.

7. The catheter according to claim 6, wherein the nerve stimulation signal is a current pulse signal, in which positive and negative currents alternately repeat during one cycle, so that in a current pulse signal of a first cycle, positive current is generated first, followed by negative current, and in a current pulse signal of a second cycle, negative current is generated first, followed by positive current, and the current pulse signals of the first cycle and second cycle are periodically repeated.

8. The catheter according to claim 7, wherein
the control unit includes:
a target scan mode unit, which generates the scan/detection control signal and changes position of the catheter and frequency or waveform of the nerve stimulation signal according to reaction sensitivity of a target nerve; and
a denervation determination unit for determining whether to excise the target nerve by receiving a detection signal from the outside for reaction of the nerve in response to the nerve stimulation signal emitted by the electrode part.

9. The catheter according to claim 8, wherein
the control unit further includes:
an excision position marking unit for marking an excision position of the target nerve according to target nerve excision determination of the denervation determination unit; and
a denervation mode unit for generating a denervation control signal according to the target nerve excision determination of the denervation determination unit.

10. The catheter according to claim 9, comprising:
a nerve stimulation frequency generating unit for generating the current pulse signal according to the control signal of the target scan mode unit and transmitting the control signal to the electrode part so as to enable detection of reaction of the target nerve; and
a denervation frequency generating unit for generating the denervation RF frequency according to the control signal of the denervation mode unit and transmitting the denervation RF frequency to the electrode part so as to enable excision of the target nerve.

11. A renal denervation catheter, comprising:
a catheter housing;
ring-shaped electrode parts, which are arranged in a ring type at preset intervals within a predetermined width in the circumferential direction of the housing and include heating electrodes for emitting a denervation RF signal so as to excise a target nerve of a kidney, a frequency generating unit for generating the denervation RF signal and transmits the denervation RF signal to the ring-shaped electrode parts; and
a control unit for generating a control signal so as to enable generation of the denervation RF signal.

12. The catheter according to claim 11, wherein the ring-shaped electrode part is divided into at least three sectors, and each of the sectors has a heating electrode disposed so as to excise the target nerve of the kidney.

13. The catheter according to claim 12, wherein a plurality of ring-shaped electrode parts are arranged at predetermined intervals in the longitudinal direction of the catheter housing.

14. The catheter according to claim 13, wherein the plurality of ring-shaped electrode parts are arranged so that positions of the heating electrodes disposed on each of the ring-shaped electrode parts are not the same as each other.

15. A renal denervation catheter, comprising:
a catheter housing;
ring-shaped electrode parts, which are arranged in a ring type at preset intervals within a predetermined width in the circumferential direction of the housing, and include detection electrodes for emitting a nerve stimulation signal so as to detect reaction of a nerve in a target nerve area of a kidney and heating electrodes for emitting a denervation RF signal so as to excise a target nerve of a kidney; and
a control unit for generating a control signal so as to enable generation of the nerve stimulation signal and the denervation RF signal.

16. The catheter according to claim 15, wherein the ring-shaped electrode parts are arranged, so that the detection electrodes and the heating electrodes are alternately repeated, and are divided into at least three sectors, respectively.

17. The catheter according to claim 16, wherein
the control unit includes:
a target scan mode unit, which generates a scan/detection control signal and changes position of the catheter and frequency of the nerve stimulation signal according to reaction sensitivity of a target nerve;
a target area determination unit for determining an excision area of the target nerve by receiving a detection signal from the outside for reaction of the nerve in response to the nerve stimulation signal emitted by the detection electrode;
a denervation mode unit for generating a denervation control signal according to excision area determination of the target area determination unit; and
a mode setting unit for controlling mode switching of the target scan mode unit and the denervation mode unit.

18. The catheter according to claim 17, wherein
the target scan mode unit includes:
a first target scan mode unit for allowing a pair of first and second detection electrodes to emit first and second nerve stimulation signals to the target nerve and to detect reaction of a nerve adjacent to a first heating electrode disposed between the first and second detection electrodes;
a second target scan mode unit for allowing a pair of second and third detection electrodes to emit first and second nerve stimulation signals to a target nerve and to detect reaction of the nerve adjacent to a second heating electrode disposed between the second and third detection electrodes; and
a third target scan mode unit for allowing a pair of first and third detection electrodes to emit first and second nerve stimulation signals to a target nerve and to detect reaction of the nerve adjacent to a third heating electrode disposed between the first and third detection electrodes.

19. The catheter according to claim 18, wherein each of the first and second nerve stimulation signals is a current pulse signal, in which positive and negative currents alternately repeat during one cycle, so that in a current pulse signal of a first cycle, positive current is generated first, followed by negative current, and in a current pulse signal of a second cycle, negative current is generated first, followed by positive current, and the current pulse signals of the first cycle and second cycle are periodically repeated.

20. The catheter according to claim 19, wherein the second nerve stimulation signal is emitted to the target nerve in a discharge section of the first nerve stimulation signal.

21. The catheter according to claim 20, wherein a plurality of ring-shaped electrode parts are arranged at predetermined intervals in the longitudinal direction of the catheter housing.

22. The catheter according to claim 21, wherein the plurality of ring-shaped electrode parts are arranged so that positions of the heating electrodes and the detection electrodes disposed on each of the ring-shaped electrode parts are not the same as each other.

23. The catheter according to claim 22, wherein
the mode setting unit includes:
a detection/excision/fixation mode unit for detecting and excising the target nerve while fixing the ring-shaped electrode part; and
a detection/excision/movement mode unit for detecting and excising the target nerve while moving the ring-shaped electrode part along a renal vessel, so as to disperse a nerve excision area.

24. The catheter according to claim 17, comprising:
a nerve stimulation frequency generating unit for generating the current pulse signal according to the control signal of the target scan mode unit and transmitting the control signal to the detection electrode so as to enable detection of reaction of the target nerve; and
a denervation frequency generating unit for generating the denervation RF frequency according to the control signal of the denervation mode unit and transmitting the denervation RF frequency to the heating electrode so as to enable excision of the target nerve.
